(19) United States
(12) Patent Application Publication
(10) Pub. No.: US 2015/0233858 A1
(43) Pub. Date: Aug. 20, 2015

(54) COMPOSITE WITH INTEGRAL SENSOR AND METHOD

(71) Applicant: Raytheon Company, Waltham, MA (US)

(72) Inventors: Julie N. Strickland, Frisco, TX (US); Brian D. Pautler, The Colony, TX (US)

(21) Appl. No.: 14/529,260

(22) Filed: Oct. 31, 2014

Related U.S. Application Data
(60) Provisional application No. 61/941,872, filed on Feb. 19, 2014.

(51) Int. Cl. G01N 27/20; G01N 33/36; H01L 31/048; D06M 15/19; D06M 15/55

(52) U.S. Cl. CPC G01N 27/20; D06M 15/19; D06M 15/55; H01L 31/048; G01N 33/36

(57) ABSTRACT

A reinforced plastic composite structure includes multiple layers, with at least one layer including an electrically-conductive element incorporated in a fabric substrate. A fabric substrate with an electrically-conductive element can be referred to as an e-textile. The electrically-conductive element of the electrically-conductive fabric layer is connected to a controller for analysis of the fabric's changing electrical properties over time as a way to identify and prevent failure of the composite structure.

### FIELD OF THE INVENTION

The invention is concerned with a composite structure with an integral sensor and a method of making such a structure.

### BACKGROUND

Reinforced plastic composites are typically manufactured with layers of fabric, such as fiberglass and carbon fiber, and liquid plastic resins in a labor-intensive fabrication process. The resin hardens to form a solid structure. The resulting composite can have any of a wide variety of shapes. But it is difficult to determine the structural integrity of the composite once it has been made. Since an operator generally is required to make the composite, variations and defects can easily occur during the fabrication process and not be noticed during fabrication.

Important structural members often are made of reinforced plastic composites for use in aircraft, ground vehicles, missiles, rockets, radomes, and vehicles used in space. Because of their structural importance (wings on an aircraft, for example), several attempts have been made to measure and monitor the strength of the composite. Traditional electronic sensors have been used in such composites, example, traditional strain gauges have been embedded in composites to measure and monitor strain in the composite.

### SUMMARY

In contrast to traditional sensors that are surface-mounted or encapsulated in a plastic composite, the present invention provides a composite with a textile layer having integral traces or sensors as part of a composite structure. An exemplary trace is a conductive thread woven or sewn into a fabric substrate. The difference in resistance or other properties of the traces, or other sensor signals, can be used to reveal stresses, cracks, or other structural anomalies.

Since many reinforced plastic composites include a layer of fabric, replacing a fabric layer with an e-textile requires minimal change to assembly practices and may have a profile as low as the previous fabric layer. Thus, added strain may be reduced or eliminated from components with a higher profile than the previous fabric layer. This means that few if any additional problems are created in the substitution of the e-textile for traditional strain gauges or other previous solutions.

More specifically, the present invention provides a composite structure, comprising multiple layers of material, at least one layer including an electrically-conductive element incorporated in a fabric substrate.

The foregoing and other features of the invention are hereinafter fully described and particularly pointed out in the claims, the following description and the annexed drawings setting forth in detail one or more illustrative embodiments of the invention. These embodiments, however, are but a few of the various ways in which the principles of the invention can be employed. Other objects, advantages and features of the invention will become apparent from the following detailed description of the invention when considered in conjunction with the drawings.

### BRIEF DESCRIPTION OF THE DRAWINGS

FIG. 1 is a schematic illustration of a system provided by the present invention incorporating a composite, a portion of which is removed to show an embedded e-textile coupled to a controller.

FIG. 2 is a schematic perspective view of section of a composite provided by the invention having multiple fabric layers, including an e-textile layer coupled to a controller.

FIG. 3 is a schematic view of an e-textile fabric layer coupled to a contoller.

### DETAILED DESCRIPTION

The present invention provides a composite material and method where one or more layers of fabric are replaced with e-textiles to create a composite with integral sensors. One characteristic of e-textiles is that one or more electrical properties, such as resistance, changes as the e-textile is bent or stretched. Therefore by measuring the resistance or other changing property, anomalies in the composite can be easily identified.

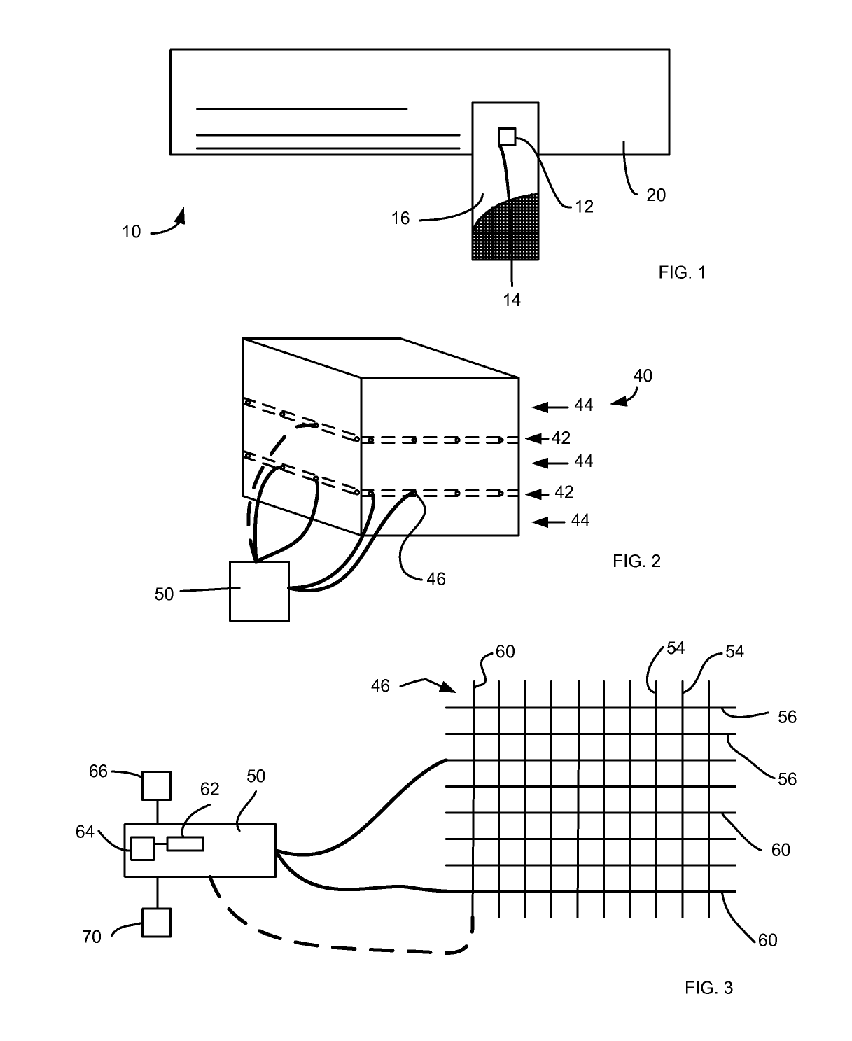

A schematic illustration of a system 10 provided by the present invention is shown in FIG. 1. In this embodiment, the system 10 includes a controller 12 connected to an e-textile layer 14 in a composite in the form of a wing 16 of an aeronautical vehicle 20, such as an airplane or a missile. A portion of the wing is removed to show the embedded e-textile layer.

A schematic cross-section of a composite 40 provided by the invention is shown in FIG. 2, and includes multiple layers of fabric 42 and plastic 44 that form a structural element. At least one layer of fabric 42 includes an e-textile 46, and the e-textile is depicted as connected to a controller 50. A dashed line from the controller 50 to another fabric layer 42 is included to show the controller 50 can be coupled to one or more than one layer in the composite 40.

The plastic layers 44 of the composite 40 may be applied in a liquid state to the fabric layer or layers 42 or over a mold and then cured or otherwise treated to form a solid structure. Consequently, the plastic can flow into and through the fabric layer 42, embedding the fabric in the plastic 44. Exemplary plastic materials can include an epoxy, such as Glycidylamine; a thermosetting plastic, such as a vinyl ester or a polyester; or a thermopastic, such as polyurethane or polypropylene; or a combination thereof.

The fabric layer 42 adds structural strength to the plastic 44. When the fabric layer 42 is an e-textile 46 the e-textile may enable monitoring of stresses in the composite 40.

Electronic textiles (often referred to as e-textiles) are fabrics that have electronic elements woven into the substrate textile. Traditional electronic elements used in e-textiles can include passive electrical components, such as conductors and resistors, and active electronic components, such as diodes and transistors.

The e-textiles used in the present application may be made from a variety of substrate and conductive thread materials. A conductive thread may be incorporated into a fabric substrate to allow current to flow. Conductive fibers used in the e-textiles provided by an embodiment of the invention can be made of metal, other materials coated with metal, metal coated by another material, or another material embedded with conductive particles, such as metal particles. The metal in these fibers can be shaved from a larger wire, bundle-drawn from a larger diameter wire, cast from molten metal, or grown around a seed material, such as a carbon fiber. Bundle-drawn metal fiber can be produced in sizes smaller than one micrometer in diameter. The conductive fibers may be “printed” on a substrate.

Metals generally form a stiff material, which is generally undesirable for textile fibers, particularly since textiles have traditionally been used for clothing, and clothing fibers are subject to stretch and bending forces during use. Metal wires tend to deform and break under repeated stretching and bending, undesirable qualities in an article of clothing.

Traditional textile materials generally used to form the fabric substrate include cotton, nylon, polyester, fiberglass, wool, etc.; in the form of elongated strands or fibers. These traditional textile materials typically are considered to be electrical insulators rather than electrical conductors. Other textile fibers can include nylon, polyester, or aramids, including Kevlar and Nomex, and polypropylenes. Each material has advantages and disadvantages. For example, nylon becomes brittle in low temperatures, while Nomex is durable for high temperature applications, since it can withstand up to 400° C.

The yarn or thread used to form the substrate can be made from one or more of any of the above conductive or non-conductive materials. The substrate may be formed similar to a shirt or other cloth material, which may be sewn from a variety of raw materials. In some embodiments, the substrate may be formed by any other textile methods, such as weaving, knitting, crocheting, knotting, pressing fibers together, or overlying fibers.

As shown in FIG. 3, the e-textile 46 may look substantially similar to a layer of fabric 42 with threads 54 and 56 running in multiple directions and crossing multiple other threads along the way. An embodiment of the present invention uses e-textiles with integral traces or sensors to create composites which will then have these traces or sensors embedded within them. One or more of the threads 54 and 56 include a conductive fiber 60 that conducts electricity, making the resulting fabric an e-textile. An exemplary composite 40 includes an e-textile 46 having a conductive thread 60 woven or sewn into a fabric substrate for use as a sensor. One or more of the conductive threads are depicted as electrically connected to the controller 50 to monitor the electrical signature, such as its resistance, of the e-textile 46 over time.

The e-textiles 46 described can be used to monitor stress and strain in a composite, structurally reinforce the composite, provide static dissipation, act as a heating element, or provide signal and power transfer. The conductive fibers used in the present application may be as thin as or thinner than the fibers of the fabric substrate. The conductive fibers can be used in existing textile machinery in typical textile processes, such as weaving, knitting, braiding, etc. In some embodiments, the conductive fibers 60 may be thicker than the fibers 54 and 56 of the fabric substrate.

A comparison of changes in the electrical signature in the e-textile fabric over time can be used to identify anomalies in the composite structure. The difference in measured resistance or other properties of the traces or sensors reveals stresses, cracks, or other structural anomalies.

The controller 50 can be configured to receive and analyze a signal from the e-textile 46. The controller 50 typically includes a processor 62, such as a microprocessor, a memory 64, and related software that configure the controller 50 to carry out its functions. The controller 50 also can include an output device 66 to output results, in either an audio or video format, such as a speaker or a monitor, respectively. The controller 50 can further include an input device 70, such as a touch screen, a pointing device (such as a pointing device commonly referred to as a “mouse”), or a keyboard. The controller 50 can be permanently positioned relative to the composite 40 for continuous or intermittent monitoring, and conducting analysis in real time or storing the data for later retrieval, or the controller 50 can be connected to the composite 40 as needed to check the condition of the composite 40.

Anomalies identified by changes in resistance in the e-textile layer of the composite structure can be used to alert an operator that further investigation is required or to indicate a potential failure. The composite structure can then be evaluated, reinforced, repaired, or taken out of service prior to a critical failure.

Measured resistance of a cable or e-textile product is essentially constant between connectors. Estimating the electrical properties of conductive e-textile fabric before it is developed into a product is difficult, since the length and width may vary and handling the fabric during the manufacturing process could inadvertently bend or stretch the fabric, changing one or more electrical properties, such as resistance. For example in one instance stretching a conductive fabric changed the measured resistance from 10 to 20 ohms. While the variation in resistance in response to stretching or bending is what allows e-textiles to be used as sensors to measure anomalies such as distortions, tears, or wrinkles.

Accordingly, in summary the present invention provides a reinforced plastic composite structure that includes multiple layers, with at least one layer including an electrically-conductive element incorporated in a fabric substrate, which can be referred to as an e-textile due to its inclusion of the electrically-conductive element. The electrically-conductive element of the electrically-conductive fabric layer is connected to a controller for analysis of the fabric's changing electrical properties over time as a way to identify and prevent failure of the composite structure.

Although the invention has been shown and described with respect to a certain preferred embodiment, it is obvious that equivalent alterations and modifications will occur to others skilled in the art upon the reading and understanding of this specification and the annexed drawings. In particular regard to the various functions performed by the above described components, the terms (including a reference to a “means”) used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments of the invention. In addition, while a particular feature of the invention can have been disclosed with respect to only one of the several embodiments, such feature can be combined with one or more other features of the other embodiments as may be desired and advantageous for any given or particular application.

What is claimed is:

1. A composite structure, comprising:
multiple layers of material, at least one layer including an electrically-conductive element incorporated in a fabric substrate.

2. A composite as set forth in claim 1, where the multiple layers include a plastic material.

3. A composite as set forth in claim 2, where the plastic includes one or more of an epoxy, a thermosetting plastic, and a thermoplastic.

4. A composite as set forth in claim 1, where the fabric substrate includes interwoven fibers.

5. A composite as set forth in claim 1, where the electrically-conductive element is embedded in the fabric substrate.

6. A composite as set forth in claim 1, where the electrically-conductive element is bonded to the fabric substrate.

7. A composite as set forth in claim 1, where the electrically-conductive element is interwove into the fabric substrate.

8. A composite as set forth in claim 1, where the conductive element includes a resistor, a transistor, a diode, or a solar cell.

9. The e-textile of claim 1, wherein the fabric substrate includes one or more of fiberglass, carbon fiber, aramid fiber, and a natural fiber.

10. A composite as set forth in claim 1, where the fabric substrate is embedded in a plastic matrix.

11. A composite as set forth in claim 1, further including:
a trace or a sensor, integral with the fabric substrate.

12. A composite as set forth in claim 1, where the electrically-conductive element is configured to communicate with a controller.

13. A composite as set forth in claim 1, where the electrically-conductive element is configured to modify an electrical signal passing through the electrically-conductive element upon a change in a structure of the fabric substrate.

14. A composite as set forth in claim 13, where resistance of the electrically-conductive element is modified to modify the electrical signal upon the change in the shape of the fabric substrate.

15. A method of testing a structure for anomalies, including:
connecting a controller to an e-textile formed in a composite structure;
measuring an electrical property of the e-textile at a first time;
measuring the electrical property of the e-textile at a second time; and
detecting anomalies based on a comparison of the measurement at the first time to the measurement at the second time.

16. A method as set forth in claim 15, wherein the electrical property is resistance of at least a portion the e-textile.

17. A method as set forth in claim 15, further including:
calculating stress or strain, of at least a portion of the composite structure, based on the comparison of the measurement at the first time to the measurement at the second time.

18. A method as set forth in claim 15, further including:
indicating potential failure of the composite structure based on the comparison of the measurement at the first time to the measurement at the second time.

19. A method as set forth in claim 18, further including:
removing the composite structure from service based on the indicated potential failure.

20. A method of integrating a sensor in a structure, including:
providing a e-textile; applying a plastic portion in a liquid state to the e-textile; and
forming a solid structure including the e-textile integrated into the plastic portion.
